(19) United States
(12) Patent Application Publication
(10) Pub. No.: US 2023/0255801 A1
(43) Pub. Date: Aug. 17, 2023

(54) HAND PROSTHESIS

(71) Applicant: PRO/BIONICS S.A. DE C.V., Tlalnepantla de Baz (MX)

(72) Inventor: Luis Armando BRAVO CASTILLO, Tlalnepantla de Baz (MX)

(73) Assignee: PRO/BIONICS S.A. DE C.V., Tlalnepantla de Baz (MX)

(21) Appl. No.: 18/015,870

(22) PCT Filed: Apr. 8, 2021
(86) PCT No.: PCT/MX2021/050018

§ 371 Date: Jan. 12, 2023

(30) Foreign Application Priority Data
Jul. 13, 2020 (MX) MX/A/2020/004609

(51) Int. Cl. A61F 2/58

(52) U.S. Cl. CPC A61F 2/588; A61F 2/585; A61F 2/586; A61F 2002/701

(57) ABSTRACT

The invention relates to a hand prosthesis that can be adapted at the forearm or wrist. The prosthesis including a mechanical design with a smaller number of parts than the prior art, thus facilitating the assembly, maintenance and production thereof. Likewise, the prosthesis includes a plurality of motion transmission systems in a housing similar to the volume of a biological hand and provides sufficient grip strength to reincorporate the user into everyday activities. The invention includes an adult hand prosthesis, a child hand prosthesis and a wrist that allows the user to hold heavy loads while moving around.

### FIELD OF THE INVENTION

The present invention generally relates to the field of mechanics and electrics and, more particularly, to the field of hand, wrist and forearm prostheses, at least partially, for adults and children, wherein the prosthesis includes electrical and mechanical elements and combinations thereof. Moreover, there is provided a hand and/or wrist prosthesis that may be subjected to relatively high loads in a fully downwardly extended arm position.

### BACKGROUND OF THE INVENTION

It has been found in the art that flexion for gripping an object, including gripping of heavy objects using the wrist and/or forearm is the movement that is most used by people with an upper limb amputation. Thus, there are many hand prosthesis designs that range from the oldest ones based on a mechanical hook mechanism to more sophisticated and complex ones with robotic movements and several micro-motors for each finger, including its phalanges and joints. These more sophisticated prostheses seem to overcome the problem of bringing the patient back to a higher quality of life and even back to working life. However, in addition to their relatively high weight, it has been found that these kind of prostheses are expensive, and because of their complexity in controlling them, hand prostheses may provide limited movement and strength needed for patients to go back to the everyday life. Furthermore, the manipulation of these prostheses is equally complex, since the high number of degrees of freedom they offer makes it difficult for an average patient to operate such prostheses.

Therefore, various prosthetic designs have been created to address this need, such as U.S. Pat. No. 8,021,435 B2, which discloses a clamp-like grip design with two upper fingers (fingers two and three) and one lower finger (finger one) actuated by a worm screw system providing sufficient force for the patient to be able to carry relatively heavy objects; however, the mechanical design causes the final prosthesis to have non-anthropomorphic dimensions making it longer than a biological limb because the location of the actuator at least partially occupies the space of the biological forearm. Additionally, in Patent No. EP 1962737 B1, the use of a pivot-based drive mechanism allows a patient to actuate movement of fingers two and three towards finger one in an anthropomorphic clamp-like grip.

Among other techniques, a reliable way employed for patient-prosthesis interaction involves myoelectricity, which essentially detects the electrical impulses generated when a muscle is voluntarily moved, wherein each impulse and/or pattern of impulses is given an interpretation to thereby reflect as an output a mechanical movement of the prosthesis through any of its degrees of freedom. This technique has proven to be quite useful, since users interact with their prosthesis through voluntary contractions of at least one muscle of the body, which is relatively simple for users, and the number of false activations (activation of the prosthesis without the user wanting it) is minimized. Therefore, a myoelectric prosthesis design is desirable whose mechanical design provides a strong grip of at least a 10 kg load in a lightweight product, with dimensions that do not exceed the dimensions of an average biological hand, and whose electrical/electronic design allows for friendly operation and that may be fitted to the different body parts (body muscles), thus allowing a patient to improve his/her quality of life.

In this line of thinking, it has also been found that prostheses using this myoelectric technology have been useful in young patients, where an easy handling of the prosthesis results in a great advantage. However, it is important to provide a mechanism that allows for easy use while being safe for children, since there are prostheses whose grip force in a flexion movement may hurt a person if it is not properly controlled, so it is desirable to design a prosthesis for children that prevents a user from improperly using the prosthesis force. Accordingly, it is desirable to provide an electromechanical hand prosthesis for children, where activation of the hand prosthesis under simple control methods is safe for the children and their environment.

Therefore, it has also been found that a prosthesis providing relatively high (greater than 10 kg) loads for the user presents other types of issues. For example, when a user or patient is carrying a heavy object while moving, then the object tends to swing based on the user movement. This swinging or pendulum-like movement causes great efforts in the wrist part, sometimes resulting in fractures of the wrist material, so it is desirable to design a wrist for a hand prosthesis that allows for carrying heavy objects, thereby preventing a wrist from suffering fractures when the user moves while carrying heavy objects.

### SUMMARY OF THE INVENTION

The present invention claims methods, apparatuses, assemblies, systems and/or devices related to upper limb prostheses, wherein the design of said prosthesis allows for modularity adaptable to the amputation level. In an embodiment of the invention, there is provided a myoelectric hand, wrist and/or forearm prosthesis. In an embodiment of the invention, there is provided a hand prosthesis wrist mechanism which allows for carrying heavy objects by gravity while a user or patient walks, that is, while the object experiences the natural wave-like movement of the arm when a user or patient walks or moves.

In an embodiment of the invention, there is provided a hand prosthesis comprised by at least one prosthesis support defined by an elongated stiff part having two sides relative to an axis of symmetry, with a thickness and two faces. Said elongated stiff part comprises on a first side of the axis of symmetry and on one of its faces at least a first flute in a longitudinal direction, that is, from the axis of symmetry to the distal end, at least partially; and at least a second flute perpendicular to the first flute. In an embodiment of the invention, the second flute reaches at least one of the ends and/or edges of the stiff part relative to the transverse axis.

In an embodiment of the invention, the elongated stiff part comprises machining marks such as by milling, for drilling, hollowing, slotting and/or combinations thereof formed on a first side of the stiff part relative to the axis of symmetry. Additionally, the same machining marks are also formed on a second side, that is, on a second side relative to the traverse axis of symmetry of said stiff part, in a mirror configuration of the machining marks formed on the first side with the machining marks formed on the second side, either on a front and/or rear face of said elongated stiff part. In a particular embodiment, the mirror configuration is a partially mirror configuration.

In another embodiment of the invention, a method of manufacturing a prosthesis support from a single elongated stiff part having a axis of symmetry is provided, which comprises: mechanical means to perform, on the elongated stiff part, at least one machining mark selected from the list of: fluting, recessing, hollowing, drilling and/or combinations thereof, so that the machining marks formed on one side of the axis of symmetry are substantially the same as the machining marks formed on the other side of the axis of symmetry; mechanical means to make at least one fold on the elongated stiff part to form a “U” shape, thus defining side walls and a central portion; mechanical means to drill at least one hole or perforation in the central portion of the “U” shape, wherein the mechanical means for folding the elongated stiff part allow the folding angles to have a radius of less than 10 mm.

In a particular embodiment, the machining marks are formed on the front and rear faces of the elongated stiff part.

In an embodiment of the invention, a hand prosthesis assembly is provided, which comprises a U-shaped prosthesis support or housing having longitudinal flutes on the inner faces of the “U” shape, an electric actuator, a screw-nut system that converts rotational motion into linear motion in the nut or movable element, a system of clamps or fingers receiving the linear motion and generating, with the use of a pivot, an opening and closing movement of the fingers also referred to as clamps. In a particular embodiment, the nut or movable element of the screw-nut system comprises at least one protrusion or protuberance having a projection that is inserted into the corresponding flutes of the support, thus locking the rotation so as to ensure a linear motion, that is, the longitudinal flutes operate as inner guides of the prosthesis support. In an embodiment of the invention, the protrusion or protuberance comprises a cylindrical portion and at its distal end the projection is defined by a straight guide coupled to the internal channels of the prosthesis support. The screw-nut relationship comprises a plurality of features such as string type, number of strings, screw length, nut stroke, etc.

In an embodiment of the invention, the electric actuator is an adjustable or non-adjustable speed electric motor producing a rotational motion. In another embodiment of the invention, the electric actuator is an actuator producing a linear motion.

The clamps or fingers being stiff or semi-stiff elongated parts having at least one substantially curved shape, at least partially.

In an embodiment of the invention, the hand prosthesis comprises two axes, a motor axis and an operating axis. The motor axis defined by a motor shaft having a first gear coupled to the motor shaft, and the operating axis comprising a second gear operationally coupled to the first gear, such that the motor axis and the operating axis are parallel relative to each other and spaced apart a distance proportional to the dimensions of the first and second gear, thus transmitting the motion from the motor to the operating axis. Additionally, the screw base is coupled to the second gear so that the screw rotates together with the second gear.

In an embodiment of the invention, the second gear design comprises a head or substantially cylindrical protrusion allowing the second gear to couple to the hole of the inner portion of the “U” shape, thus providing a concentric relationship between the second gear and the support hole. In an embodiment of the invention, there is provided a bearing coupled between the support hole and the cylindrical protrusion of the second gear to facilitate the rotation of the second gear around the operating axis. In a particular embodiment, the bearing is pressed at one of its ends by an external pressing part comprising adjustable fastening mechanical means such as a screw system located outside the hole of the “U” shape. Thus, the external pressing part exerts pressure on the bearing, resulting in an adjustment of the motion or vibrations experienced by the bearing during operation of the prosthesis.

In an embodiment of the invention, the clamps are comprised by at least two sets of elongated elements (each set comprising at least one elongated element) corresponding to finger one and fingers two and three, respectively, one finger being concave and one finger being convex that may or may not be symmetrical relative to the operating axis, whose configuration allows for an opening or closing movement from a linear motion, through a pivot on the support or housing. In an embodiment of the invention, the proximal portion of each clamp comprises a slide or slot having circular ends with a diameter matching the diameter of the cylindrical protrusions, such that the cylindrical portion of the protuberance or cylindrical protrusion is coupled to the slide, and wherein the middle portion of each set of clamps is coupled, by at least one joint, to the support that in this case operates as a fixed or reference part for pivoting of each set of clamps. Thus, when the nut or movable element of the screw-nut system is moved, depending on the rotation direction of the screw, the nut or movable element generates an opening or closing movement of the clamps, with said clamps having a pivot or rotation relationship with the prosthesis support. In an embodiment of the invention, the location of each clamp pivot and/or the features of the screw-nut system may vary so that the resulting opening and/or closing movement of the clamps may also vary and/or be intended for a specific application.

In another embodiment of the invention, there is provided a prosthesis having elastic mechanical means coupled to the clamps or fingers, such that said elastic mechanical means maintain a constant force on said fingers or clamps to keep said fingers or clamps in an extended, flexed or intermediate position. In a particular embodiment, the force applied by said elastic mechanical means causes the fingers or clamps to flex. In an alternative embodiment, the force applied by said elastic mechanical means causes the fingers or clamps to extend. In an alternative embodiment, the force applied by said elastic mechanical means causes the fingers or clamps to remain in an intermediate position. Additionally, an electric actuator is coupled to a mechanism providing a linear motion to a movable element, such that when said movable element is moved in one direction said movable element pushes the fingers or clamps so that the fingers or clamps are extended or opened. Thus, there is provided a hand prosthesis having at least two sets of fingers corresponding to finger one and fingers two, three, four and/or five, wherein an elastic means generates the flexion movement, and an actuator generates an extension movement by moving forward in one direction, thereby overcoming the resistance generated by the elastic means. Thus, when the actuator returns (by moving in the opposite direction) the elastic means work so as to cause the mechanical flexion of the hand, wherein the flexion speed of the hand may be regulated with the actuator returning speed. Thus, the flexion force is not dependent upon an actuator but upon an elastic means that may be replaced or adjusted to vary the resulting flexion force being applied. One skilled in the art will appreciate that the mechanical properties of said mechanical means may vary depending on the application and gripping force desired for the prosthesis, without affecting the subject matter of the present invention. Thus, elastic means with a specific Young's modulus may generate a gripping force, i.e. flexion, and other elastic means with another Young's modulus may generate another gripping force in addition to other mechanical properties. Additionally, coatings and/or non-slip covers are provided anywhere in the prosthesis for ease of gripping. Said coatings and/or covers being able to provide an anthropomorphic shape.

In a particular embodiment, the elastic means is a torsion spring. However, one skilled in the art will appreciate that the mechanical element used as an elastic means may vary without affecting the subject matter of the present invention, and that said elastic means may also be elastic bands or springs of different types, such as compression springs, extension springs, torsion springs or combinations thereof, etc. In an alternative embodiment, the electric actuator generates the flexion movement of the fingers or clamps, and the elastic means generates the extension movement of the fingers or clamps.

In an embodiment of the invention, a prosthesis support is provided from a single elongated part including milling marks of different types, which allow the fastening, retaining, attachment and/or electronic elements of the prosthesis to be at least partially coupled to and submerged in the support thickness, without hindering or affecting the free movement of the prosthesis movable parts. One skilled in the art will appreciate that the fastening, retaining, attachment and/or electronic elements may vary in shape, size and number without affecting the subject matter of the present invention, and that said elements may also include nuts, rivets, screws, bolts, PCB cards, end/start of stroke switches, sensors, etc.

In an embodiment of the invention, the hand prosthesis uses at least one mechanical transmission to transmit the motion from the motor to the fingers to cause said fingers to open and close. In an embodiment of the invention, a first transmission is coupled through a gear system engaging the motor shaft with the screw in an inverted position or reverse direction between the motor shaft and the screw. Thus, the screw axis and the motor axis are parallel and non-concentric relative to each other, with the shaft and the screw tip pointing out to different directions, that is, the motor shaft points out to the proximal portion of the prosthesis (to the stump or biological part of a patient) and the screw tip points out to the distal portion. In an alternative embodiment, the motor shaft points out to the distal end of the prosthesis.

In an embodiment of the invention, the gear system is defined by spur gears. In a particular embodiment, the gear system is defined by spur gears with helical teeth. One skilled in the art will appreciate that the way in which the gears are coupled to the axes may vary without affecting the subject matter of the present invention, and that fastening means, bearings, pins, etc., may also be used. In an embodiment of the invention, the transmission of motion from the motor to the screw is performed in a 1 to 1 ratio, that is, with the same diameter or crown in the gear system. In an embodiment of the invention, the transmission is a speed reduction transmission. In an embodiment of the invention, the transmission is a speed booster transmission. Additionally, one skilled in the art will appreciate that the type of gear may vary without affecting the subject matter of the present invention, and that a bevel gear, spur gear, helical gear, bevel gear with straight teeth and/or bevel gear with helical teeth and/or hypoid bevel gear, etc. may also be used.

In an additional embodiment of the invention, the prosthesis comprises coupling articulated extensions corresponding to fingers four and five, wherein the drive gear includes a design that allows an additional transmission to be coupled in order to activate said fingers four and five. In a particular embodiment, said additional transmission is defined by a system of pulleys or transmission belts, including at least one assembled roller to change the tensioning direction of the pulleys or belts, such that when said direction is changed without affecting the pulleys or belts tensioning, said pulleys or belts may be more easily coupled and activated. In a particular embodiment, the pulley or bell is defined by an elastic band. Therefore, the motor is coupled to an external face of the U-shaped support by fastening elements manufactured on design, such that in an embodiment of the invention said design comprises additional elements so that fingers four and five may be coupled to the prosthesis body by a joint in the proximal portion (proximal phalanx) at least partially providing at least one degree of angular freedom. In a particular embodiment, fingers four and/or five have stiff middle and/or distal phalanges. In a particular embodiment, fingers four and/or five comprise at least one joint providing at least one degree of angular freedom to at least one of its phalanges. In an embodiment of the invention, when a flexion and/or extension movement of fingers four and/or five is activated, fingers four and/or five move along with fingers two and three. In a particular embodiment, fingers four and/or five comprise a delay mechanism such that, when a flexion and/or extension movement of fingers four and/or five is activated, fingers four and/or five move after the movement of fingers two and/or three. In a particular embodiment, fingers four and/or five comprise a forward mechanism such that, when a flexion or extension movement is activated, fingers four and/or five move previously to and/or independently from the movement of fingers two and/or three.

One skilled in the art will appreciate that the term clamp and/or finger refers to the prosthesis element that, when opened or closed, is used to hold objects, unless otherwise indicated.

Additionally in an embodiment of the invention, at least one retainer is used for each motor shaft and/or screw as a support for the axial load generated during movement, thereby preventing the motor shaft and/or the screw from exiting or jumping out of the motor body. In an embodiment of the invention, the retainer is the same as the PCB card accommodating some of the electronic components of the prosthesis. In a particular embodiment, the PCB card is a card with a thickness greater than or equal to 3.175 mm.

In an alternative embodiment, the type of transmission used for the transmission of motion from one element to another is selected from at least one of the list of: drive chains, drive belts, gears, friction wheels, friction discs, pins, Hooke's joints, constant velocity universal joints, camshafts and/or pulleys.

In an embodiment of the invention, the type of gear used for the transmission of motion is selected from at least one of the list of: a spur gear, bevel gear, straight teeth, single or double helical teeth, hypoid teeth, worm screw, crown, rack and/or pinion-chain.

In an embodiment of the invention, the prosthesis comprises coupling the fingers four and five. Said coupling is performed by means of a joint located at the proximal portion, i.e. the proximal phalanx, of each finger, allowing at least one degree of freedom corresponding to a transverse rotation, that is, the rotation allowing for flexion/extension of said fingers four and/or five up to a mechanical and/or electronic limit per sensor or switch. Additionally, in a particular embodiment, at least one of said fingers four and/or five further comprises at least one joint corresponding to the middle phalanx and/or distal phalanx. In a particular embodiment, the activation of the flexion/extension movement of said fingers four and five is performed by an additional transmission coupled to the drive gear or first gear of the hand prosthesis.

Additionally, in an embodiment of the invention, there is provided a hand prosthesis assembly comprising an activation mechanism consisting of an actuator and an elastic element, such as a spring. Therefore, when the actuator is activated, the actuator generates an extension movement on finger one (thumb) and the corresponding flexion movement is performed by the elastic element. Thus, there is provided a hand prosthesis comprising a hand having at least one articulated finger and at least one actuator. The at least one articulated finger comprising elastic means causing the at least one articulated finger to be in a generally closed or flexed position, and a tracking bar that, when pushed with a leverage greater than that generated by the elastic means, allows the articulated finger to rotate relative to said joint, thereby extending the articulated finger. The actuator coupled to a worm screw/movable displacement element system that, when energized, moves said movable displacement element which has attached a lever that, when coming into contact with the tracking bar of the articulated finger, extends said articulated finger, and wherein said movable displacement element only activates the tracking bar in one direction, since in the reverse direction it is the spring that moves the articulated finger. In other words, the actuator activates the extension movement, allowing the elastic means to perform the flexion movement.

One skilled in the art will appreciate that the type of spring to perform a flexion movement of finger one may vary without affecting the subject matter of the present invention, and that a torsion spring, flexion spring, etc., may also be used.

Additionally, in an embodiment of the invention a wrist prosthesis providing at least one degree of angular freedom is claimed. Said wrist prosthesis comprising a fixed link with holes at different angles and a movable link defined by a wrist housing having an at least partially longitudinally elongated tracking bar above the rotation axis of the at least one degree of angular freedom, such that the tracking bar body is located inside the housing and at least one distal end of the track bar protrudes from said housing. Said tracking bar being able to be pressed on at least a first distal end or on a second distal end, such that, when pressed on said first or second distal end, said first or second distal end moves linearly (or the tracking bar). The wrist prosthesis further comprises mechanical means to convert the linear motion into rotational motion, such as a gear-rack system, so that the rack is coupled to or becomes a part of the tracking bar middle portion, wherein the gear of the gear-rack system comprises a shaft extending in both directions along the rotation axis of said gear. Said shaft is coupled, in at least one of its ends, to the wrist housing through a bearing that facilitates the shaft rotation. The middle portion of said shaft comprising at least one cam. The shaft of the wrist further comprising mechanical means to hold a first end of a cord which is wound up in the shaft in one or another direction when the shaft is rotated. The second end of the cord pulls an arrangement of at least one pin or bolt, thereby defining an arrangement of pins. The arrangement of pins being capable of moving from the inside of the wrist housing to project in a linear displacement out of the wrist housing through at least one hole in said wrist housing, leaving its proximal end inside the wrist housing body and its distal end inside any of the holes of the fixed link so that, when the movable link is attempted to be rotated relative to the fixed link, said pin locks out the at least one degree of freedom, providing a resistance equal to the mechanical properties of the same manufacturing material and the manufacturing material of the housing, in a shear stress relationship.

In an embodiment of the invention, the pin or bolt is made of a metal or alloy, such as steel, aluminum, bronze, etc. Said arrangement of pins comprises, at its base and inside the housing body, at least one compression spring pushing said arrangement of pins out of the wrist housing. Thus, when the second end of the cord pulls the arrangement of pins to overcome the force of the compression spring, the pins almost entirely enter the wrist housing body. In an embodiment of the invention, a magnet is inwardly coupled to the wrist so that, when the pins are contracted to a maximum extent, the magnetic base of said pins comes into contact with said magnet so that the pins are magnetically contracted. By pressing the tracking bar in the opposite rotation direction, the cord ceases to pull the base of the pins and the cam physically separates the arrangement of pins from the magnet so that the compression spring(s) automatically and mechanically push and project said pins out.

One skilled in the art will appreciate that the mechanical means to convert the linear motion into rotational motion may vary without affecting the subject matter of the present invention.

### BRIEF DESCRIPTION OF THE DRAWINGS

Some of the measures and/or dimensions of the figures shown have been exaggerated for purposes of illustration.

FIG. 1 shows a diagram of a hand prostheses driven by a screw mechanism found in the art.

FIG. 2 shows a diagram of a hand prostheses driven by a screw mechanism according to an embodiment of the invention.

FIG. 3 shows an isometric view of a movable displacement element coupled to the screw mechanism according to an embodiment of the invention.

FIG. 4 shows a top view of an elongated plate without machining marks.

FIG. 5 shows a top view of an elongated plate with a plurality of different types of machining marks formed on one side relative to the axis of symmetry of said plate.

FIG. 6 shows a top view of an elongated plate with a plurality of machining marks of different types formed on both sides relative to the axis of symmetry of said plate, thereby defining a mirror configuration.

FIG. 7 shows a top view of an elongated plate with machining marks formed on the contour thereof.

FIG. 8 shows an isometric view, side view, side sectional S-S view, and front view of an elongated U-shaped folded plate with a plurality of different types of machining marks.

FIG. 9 shows a view of a partial left-hand prosthesis assembly, wherein the screw mechanism, the movable element, the motor and the transmission system are coupled to the U-shaped plate, and wherein the motor occupies the palm space corresponding to fingers four and five.

FIG. 10 shows a side view of three different positions of the clamp mechanism with one pivot for each clamp or group of clamps.

FIG. 11 shows a side view of a right-hand prostheses, wherein the clamps comprise a coating and/or surface to provide a mostly anthropomorphic shape.

FIG. 12 shows an isometric view of a transmission system drive gear defined by a gear according to an embodiment of the invention.

FIG. 13 shows an isometric view of a movable link of an adjustable wrist mechanism.

FIG. 14 shows an isometric view of a fixed link of an adjustable wrist mechanism.

FIG. 15 shows a bottom view of the movable link in three different positions, i.e. locked, released and maintained released position.

FIG. 16 shows an isometric view of the movable link with the fixed link of the adjustable wrist being in a pre-assembled position.

FIG. 17 shows the fixed link with the movable link of the adjustable wrist mechanism being already assembled, in an isometric view and a side view, in three different positions and with the movable link being rotated at least one adjustment position.

FIG. 18 shows an isometric view of the activation button mechanism within the adjustable wrist movable link which generates an angular displacement upon a displacement of the button.

FIG. 19 shows a front view of a pin folding and unfolding mechanism in an unfolded position, which is the default position.

FIG. 20 shows a front view of the pin folding and unfolding mechanism in a released position.

FIG. 21 shows a front view of the pin folding and unfolding mechanism in a maintained released position.

FIG. 22 shows a top view of a hand prosthesis with all of its elements, including fingers four and five being activated by a system of pulleys or elastic bands coupled to the drive gear.

FIG. 23 shows a side view of a hand prosthesis according to an embodiment of the invention, wherein the electro-mechanical drive mechanism only generates the extension movement while a mechanical elastic system generates the flexion movement, in a first substantially flexed position.

FIG. 24 shows a side view of a hand prosthesis according to an embodiment of the invention, wherein the electro-mechanical drive mechanism only generates the extension movement while a mechanical elastic system generates the flexion movement, in a first substantially extended position.

### DETAILED DESCRIPTION OF THE INVENTION

The following description is provided to enable those skilled in the art to make and use the embodiments disclosed herein, and it is provided within the context of a particular application and the requirements thereof. Various modifications to the embodiments disclosed herein will become easily evident to those skilled in the art and the general principles defined herein may be applied to other embodiments and applications without departing from the spirit and scope of the present disclosure. Thus, the present invention is not limited to the embodiments disclosed herein, but on the contrary, the present invention must conform to the widest scope consistent with the principles and characteristics disclosed herein.

The methods and processes described in the section entitled “Detailed Description of the Invention” may be incorporated as codes and/or data which may be stored on a computer-readable storage medium as described above. When a computer system reads and executes the code and/or data stored on a computer-readable storage medium, the computer system performs the methods and processes incorporated as data structures and code stored on the computer-readable storage medium.

Moreover, the methods and processes described herein may be included in modules and hardware devices. These modules or devices may include, but are not limited to, an application specific integrated circuit (ASIC) chip, a field programmable gate array (FPGA), a dedicated or shared processor that executes a specific software module or a piece of code at a given time and/or other programmable logic devices known so far or subsequently developed and which are hereinafter referred to as programmable elements. When said modules or hardware devices are activated, said modules or hardware devices perform the methods and processes included therein.

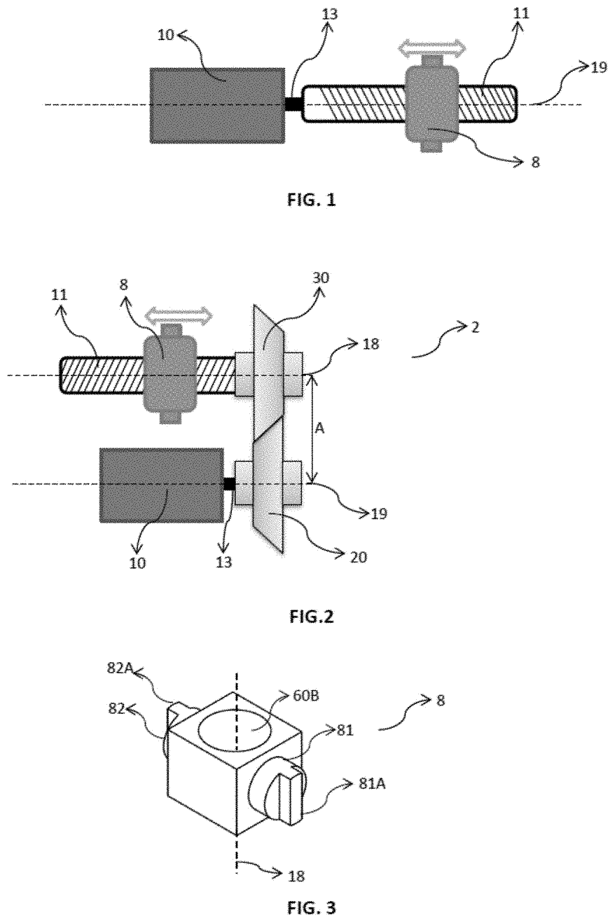

FIG. 1 shows a hand prosthesis activation mechanism comprising an arrangement of motor 10, worm screw 11 and movable displacement element 8, wherein the motor 10 and the worm screw 11 share the same motor axis or rotation axis 19. The movable displacement element 8 comprising a central hole having a thread matching the teeth of the worm screw 11, wherein the movable displacement element 8 locks the rotation relative to axis 19. The movable displacement element 8 slides linearly along axis 19, either in one direction or in the opposite direction depending on the direction of rotation of motor 10. The arrangement further comprising a motor shaft 13 pointing to the prosthesis distal end, the motor shaft 13 including mechanical elements to be operatively coupled to the worm screw 11, such that the worm screw 11 rotates in the same way that the motor 10 rotates its motor shaft 13. Therefore, in view of the stresses undergone by a prosthesis having the configuration of the previous art, there is a need for a retainer (not shown in FIG. 1) that prevents the worm screw 11 and/or the motor shaft 13 from exiting or disengaging from the mechanism by being ejected, longitudinally moving away along axis 19 as a result of said stresses.

FIG. 2 shows a hand prosthesis activation mechanism 2 comprising an arrangement of motor 10 and worm screw 11 according to an embodiment of the invention, wherein the same rotation axis is not shared. Thus, the motor rotation axis 19 is parallel to the rotation axis of the worm screw 11 or operating axis 18, but they are not collinear and/or concentric with each other, since they are spaced apart a distance M which is greater than 0. Additionally, it can be observed that the motor shaft 13 is pointing to one side, and the tip of the worm screw is pointing to the other side, that is, each one is pointing to an opposite direction. In an embodiment of the invention, the motor shaft 13 points to the prosthesis proximal end. In another embodiment of the invention, the motor shaft 13 points to the prosthesis distal end. Therefore, a mechanical transmission is used for the transmission of motion from a drive axis 19 to a driven axis 18. In an embodiment of the invention, the transmission of motion between the drive axis 19 and the driven axis 18 is performed by means of a transmission system. In an embodiment of the invention, the transmission system is defined by a gear system 20-30 defined by a drive gear 20 and a driven gear 30. In a particular embodiment, the gear system 20-30 comprises spur gears. In a particular embodiment the gear system 20-30 comprises spur gears with helical teeth. In an alternative embodiment, the gear system 20-30 comprises bevel gears with straight or helical teeth. Additionally, a movable displacement element 8 is shown, the movable displacement element 8 comprising a hole having a thread matching the worm screw 11 so that, when said worm screw 11 is rotated and the rotation of the movable displacement element 8 is restraint, the movable displacement element 8 moves along the driven axis 18, either in one direction or in the opposite direction, based on the direction of rotation of the worm screw 11, which is driven in one direction or in the opposite direction by the motor 10 through the transmission system 20-30. In an embodiment of the invention, the worm screw 11 is a worm screw having a plurality of inlets. In a particular embodiment, the worm screw 11 is a worm screw having four inlets.

One skilled in the art will appreciate that the technique for activating the motor 10 in one direction or in the opposite direction may vary without affecting the subject matter of the present invention.

FIG. 3 shows a perspective view of a movable displacement element 8 according to an embodiment of the present invention. Thus, it is shown that the movable displacement element 8 has a substantially parallelepiped shape comprising a hole 60B having a diameter and one string or a plurality of strings (not shown in FIG. 3) matching the diameter and string(s) of the worm screw 11; at least two cylindrical protrusions 81 and 82; and at least two lead ends 81A and 82A for each cylindrical protrusion, the lead ends 81A and 82A at least partially coupling to flutes formed in the prosthesis housing, such that in addition to straightly guiding the movable displacement element 8, the lead ends 81A and 82A also lock the rotation relative to the driven axis 18.

The lead ends 81A and 82A located on the cylindrical protrusions 81A and 82A respectively, each of the lead ends being defined as a nose or projection of the cylindrical protrusions 81A and 82A, wherein the lead ends 81A and 82A have an elongated or non-elongated parallelepiped shape, and wherein the longitudinal axis of the lead ends 81A and 82A is parallel and/or concentric with the driven axis 18. In an embodiment of the invention, the lead ends 81A and 82A comprise rounded corners and/or edges.

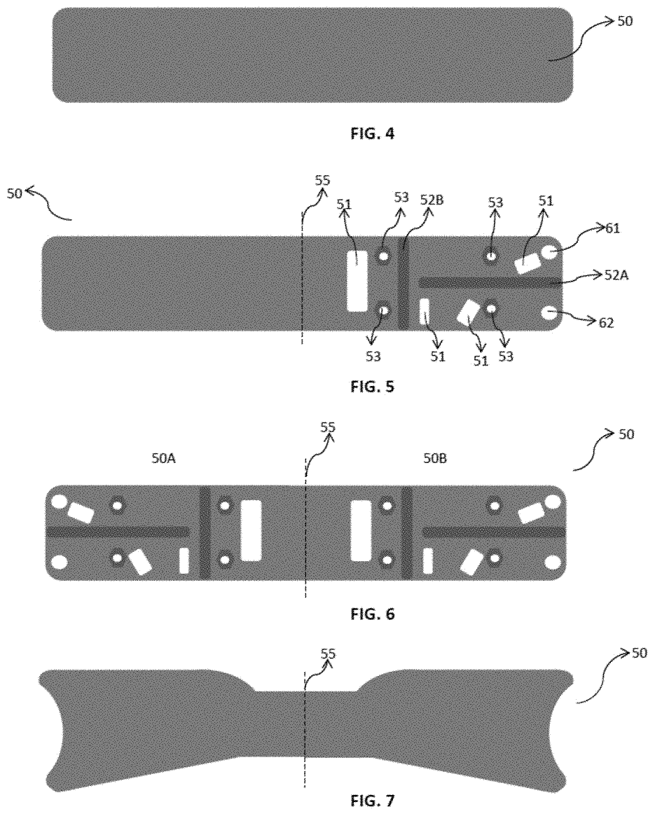

FIG. 4 shows a substantially symmetrical elongated plate 50 made of a rigid material, wherein according to an embodiment of the invention, said elongated plate 50 is subjected to machining in order to accommodate the activation mechanism 2, thus using a single piece. Therefore, in an embodiment of the invention, the elongated plate 50 is made of metal. In a particular embodiment, the elongated plate 50 is made of aluminum. In a particular embodiment, the elongated plate 50 is made a ductile, rigid material. In another embodiment of the invention, the elongated plate 50 is not symmetrical. In an embodiment of the invention, the elongated plate 50 is made of ductile aluminum, such as 1100 aluminum and/or the like.

FIG. 5 shows the elongated plate 50, which has been subjected to a plurality of machining marks 51, 52 and/or 53 on one side of its axis of symmetry 55, wherein the axis of symmetry 55 is defined by a transverse axis passing through the center thereof, i.e. a transverse axis of symmetry 55. Therefore, the machining marks 51, 61 and 62 are defined by eyelets or milled cuttings passing throughout the entire thickness of the elongated plate 50, wherein the machining marks 51, 61 and 62 may vary in shape, dimensions, number, angle, configuration, and/or location according to the requirements of prosthesis 1. The machining marks 52 are defined by hollows or milled cuttings partially passing through the thickness of the elongated plate 50, wherein the machining marks 52 may vary in shape, dimensions, number, angle, configuration, and/or location according to the requirements of prosthesis 1. In a particular embodiment, the machining marks 52 include elongated flute-like machining marks in a longitudinal and/or transverse direction relative to the elongated plate 50, such that they operate as guide or rail elements for movable parts and/or to house and/or retain elements of the prosthesis. Therefore, a particular embodiment of the invention comprises at least one elongated machining mark in a longitudinal direction on each side of the axis of symmetry 55, thus defining a longitudinal sliding channel or guide rail 52A. The machining marks 53 are defined by a combination of milled cuttings, with a part of said milled cuttings passing through the entire thickness of the elongated plate 50, and with another part of said milled cuttings partially passing through the thickness of the elongated plate 50, wherein said milled cuttings 53 may vary in shape, dimensions, number, configuration, and/or location according to the requirements of prosthesis 1. Therefore, according to an embodiment of the invention, the machining marks 51, 61, 62, 52 and/or 53 are used to house and/or hold at least one retaining element, at least one movable element of the activation mechanism 2, at least one removable or non-removable connection element, at least one electronic element, and/or at least one electromechanical element. Therefore, in a particular embodiment, the electronic element is selected from at least one of the list of: sensors, switches, dry/wet contacts, connectors, cables, etc. Additionally, in a particular embodiment, the machining marks 53 include a hollowed hexagonal shape formed through a hole in the center that allows for the accommodation of a commercial nut, such that said nut body is inserted in the body of the elongated plate 50 and a screw may be introduced to be coupled to said nut without the need to secure said nut with the use of a tool. One skilled in the art will appreciate that the machining marks 53 may vary without affecting the subject matter of the present invention, and that different types of nuts and/or mechanical removable connection elements may be used. One skilled in the art will appreciate that the thickness of the elongated plate 50 may vary without affecting the subject matter of the present invention. Additionally, one skilled in the art will appreciate that the shape, dimensions, number, configuration, and/or location of the machining marks 51, 61, 62, 52 and/or 53 may vary without affecting the subject matter of the present invention, and that the machining marks may be formed on either face of the elongated plate 50.

FIG. 6 shows the elongated plate 50, which has been subjected to the machining marks 51, 61, 62, 52 and/or 53 on both sides 50A and 50B of its axis of symmetry 55. Additionally, it can be observed how the machining marks formed on the side 50A correspond to the machining marks formed on the side 50B in a mirror configuration, thus defining the machining marks 51, 61, 62, 52 and 53, in addition to the machining marks 51′, 61′, 62′, 52′ and 53′ for the corresponding mirror configuration. Thus, with the elongated plate 50 having the same machining marks on both sides in a mirror configuration, the elongated plate 50 may be used for a left hand prosthesis or for a right hand prosthesis. In a particular embodiment, the machining marks formed on one side of the elongated plate 50 are at least partially the same as the machining marks formed on the other side of the elongated plate 50. In an embodiment of the invention, part of the machining marks are formed after said plate has been folded into a “U” shape.

FIG. 7 shows a plate 50 like that shown in FIG. 4, but the contour of the plate 50 has been subjected to machining marks according to an embodiment of the invention in order to provide a more anthropomorphic shape. One skilled in the art will appreciate that the shape, dimensions, number, location, angle, and/or configuration of the machining marks formed on the contour of the plate 50 may vary without affecting the subject matter of the present invention.

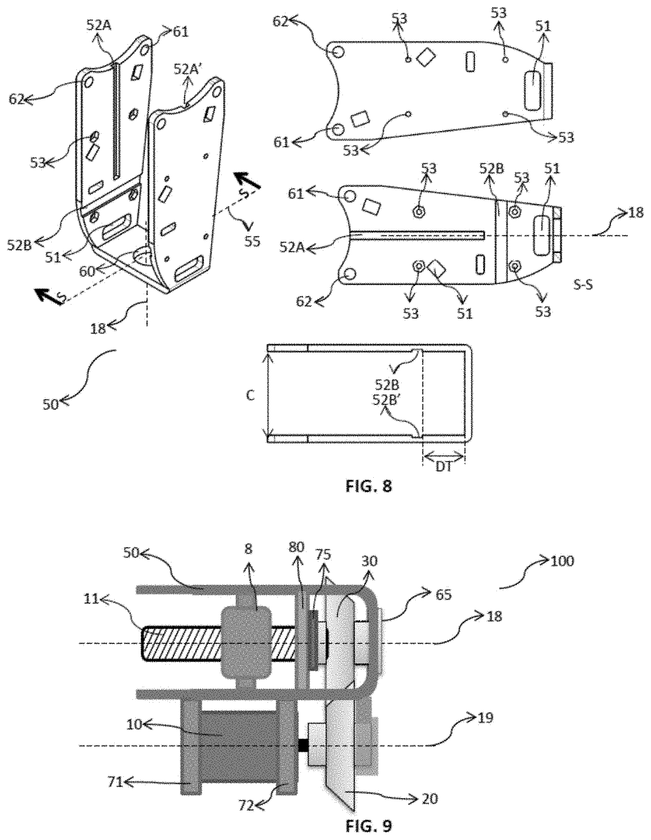

FIG. 8 shows the plate 50 in different views, such as perspective, side, side sectional S-S, and front views, wherein the contour machining marks of the FIG. 7 are maintained, as well as the machining marks 51, 61, 62, 52 and 53 (in addition to their corresponding mirror machining marks) according to an embodiment of the invention, and wherein the plate 50 has been folded into a “U” shape having a substantially circular hole 60 in the central portion of the curvature, and wherein the corner where the folding or curvature is made comprises a natural folding radius on the inner and outer face that is directly proportional to the thickness of the plate 50. Thus, a plurality of machining marks are shown on each of the inner and outer faces of the U-shaped plate. In an embodiment of the invention, the hole 60 has a diameter that allows for engagement of an axial, radial or combined bearing, wherein the driven gear 30 is aligned, seated and longitudinally rotated.

FIG. 9 shows an assembly 100 of prosthesis activation mechanism 2 with the plate 50 already folded into a “U” shape according to an embodiment of the present invention, as well as the fastening elements 71 and 72 holding the adjacent motor 10 with an outer face of one side of the U-shaped plate 50. In a particular embodiment, depending on the type of prosthesis, either a left hand prosthesis or a right hand prosthesis, and in order to feature an anthropomorphous design, the motor 10 is placed on the outer face of the plate 50 on the side where fingers four and five are located, that is, the ring and little fingers respectively, so that the volume occupied by the motor 10 substantially matches the volume of a biological hand palm. Additionally, a retainer 80 is shown, which provides a specific operating distance DT between the inner face of the plate in the center of the curvature and the retainer 80, and a fixed wall on which the transmission of motion is rested through the driven gear and the worm screw. Additionally, the retainer 80 prevents the worn screw 11 and/or the driven gear 30 from being disengaged, exiting out of the activation mechanism 2 along the driven axis 18. In an embodiment of the invention, the retainer 80 consists of a substantially rectangular flat plate made of a rigid material with a circular hole therein (not shown in FIG. 8) and whose location allows for a parallel and concentric attachment to the worm screw 11 and the hole 60, such that the worm screw 11 passes through the retainer 80 for free rotation thereof. In an embodiment of the invention, the retainer 80 is coupled to the plate 50 by means of the machining marks 52B and 52B′. That is, the retainer 80 and the plate 50 allow the engagement of the worm screw 11 to the driven gear 30 to freely rotate longitudinally in both directions, but restrain any other angular or displacement degree of freedom. Thus, the gear 30 rests and remains aligned by means of the hole 60 in the plate 50, and the worm screw rests and remains aligned on the axis 18 by means of the retainer 80 with its hole (not shown in the Figures) and movable displacement element 8 and its lead ends 81A and 82A, which move along the guide rails 52A and 52A′. Therefore, the restraint of the longitudinal rotation by means of the plate 50 results in the movable displacement element 8 and its lead ends 81A and 82A displacing in any direction along the axis 18. Additionally, a bearing 65 allowing for longitudinal rotation (relative to axis 18) is shown. The dimensions of the bearing 65 allow for engagement of the driven gear 30 with the hole 60 in the plate 50, such that the bearing 65 allows the gear to remain aligned during rotations or revolutions thereof. One skilled in the art will appreciate that the dimensions of the hole 60 and/or the gear 30 may be modified in order to match commercially available bearings. In an embodiment of the invention, the bearing 65 includes damping elements and/or misalignment tolerance. In a particular embodiment, the bearing 65 is a radial and/or combined bearing.

In an embodiment of the invention, a material allowing for sliding is coupled between the retainer 80 and the gear 30. In a particular embodiment, a bearing 75 is coupled between the retainer 80 and the gear 30 in order to facilitate the longitudinal rotation of the gear 30 relative to the retainer 80 which the gear 30 is constantly contacting. One skilled in the art will appreciate that the dimensions, materials, capacities, etc. of the bearings may vary without affecting the subject matter of the present invention. In a particular embodiment the bearing 75 is an axial and/or combined bearing. Additionally, the bearings described herein may be axial, radial or combined bearings, depending on the intended application.

One skilled in the art will appreciate that the dimensions of the lead ends 81A and 82A match the dimensions of the rails 52A and 52A′ so that, when inserted, the lead ends 81A and 82A allow for linear displacement, preventing any misalignment and/or rotation of the movable displacement element 8, that is, the rotation of the movable displacement element 8 is locked, but allowing movement thereof in any direction along said rails. Additionally, in an embodiment of the invention, the rails 52A and 52A′ and/or the lead ends 81A and 82A comprise coatings and/or treatments that minimize vibration and/or facilitate the displacement of the movable displacement element 8, either with the use of materials having a low coefficient of friction and/or with a lubricant.

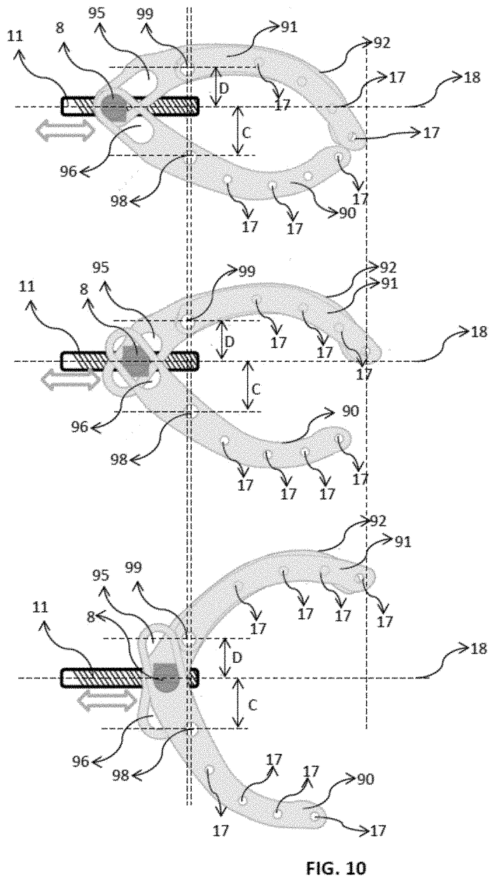

FIG. 10 shows a side view of a partial three-finger hand prosthesis defined by a mechanism provided with concave-convex claw-like clamps relative to the axis 18, driven and activated by a movable displacement element 8 according to an embodiment of the present invention, and wherein three different positions, such as closed, middle and open, are shown. Thus, a claw- or scissors-like mechanism provided with anchor points or pivots 98 and 99 is shown. Said mechanism comprising concave-convex clamps corresponding to finger one 90 (thumb), finger two 91 (index) and finger three 92 (middle) which are driven from an at least partially closed position to an at least partially open position, and vice versa. In another embodiment of the invention, said mechanism is further provided with clamps corresponding to fingers four and five. Therefore, the pivots 98 and 99 are fixed to the plate 50 (not shown in FIG. 9) and allow the clamps 90-92 to rotate relative to the pivots 98 and 99 when the clamps 90-92 are driven by the movable displacement element 8. Thus, the pivot 98 causes the clamp 90 to rotate around the pivot 98, thus generating the corresponding clamp opening or closing movement, and the pivot 99 causes the clamps 91-92 (or 91-94 in an embodiment of the invention) to rotate around the pivot, thus generating the corresponding clamp opening or closing movement. The drive to open and close the clamps 90-92 is performed by the movable displacement element 8, which is coupled on one side to the cylindrical protrusion 81, in the eyelet or slide 96 of the clamp 90, and on the other side with the cylindrical protrusion 82, in the eyelet or slide 95 of the clamps 91-92. Thus, in addition to the lead ends 81A and 82A that are coupled to the flutes 52A and 52A′, the cylindrical protrusions 81 and 82 (where the lead ends 81A and 82A are located) are coupled, in view of the dimensions thereof, to the corresponding slides 95 and 96 to directly urge clamps 90-92. Thus, in the flexed or closed position of the clamps 90-92, the movable displacement element 8 is in a position relative to the worm screw 11 (in this case on the prosthesis proximal side), so that when the worm screw 11 is rotated and the movable displacement element 8 is displaced to the distal side, the clamps 90 and 91-92 may pivot around pivots 98 and 99 to cause opening thereof as the movable elements 8 displaces. One skilled in the art will appreciate that the concave-convex relationship depends on the point of view from which it is looked. For example, clamps 90 and 91-92 have a concave-convex relationship relative to the operating axis 18.

In an embodiment of the invention, the clamp mechanism is a non-symmetrical concave-convex clamp mechanism, whose configuration allows the clamp distal ends in an at least partially closed position to nearly contact or contact each other. In an embodiment of the invention, the clamp relationship is a relationship selected from the list of: a straight, non-straight, concave-convex, curve relationship and/or combinations thereof. The slides being able to be at least partially straight and/or curved grooves provided with rounded ends with a varying length according to the specific application of the prosthesis.

Therefore, the separation distance of the pivot 98 and 99 relative to the axis 18 of the worm screw 11 is defined by the distances C and D respectively. Thus, in order to provide a mostly anthropomorphic movement, the distances C and D are not the same, such that the pivot 99 of the clamps 91-92 (index and middle finger) is closer to the axis 18 relative to the other pivot 98, so that the resulting pivotal rotation of the clamps 91-92 is greater than the pivotal rotation of the clamp 90. This can be observed in the closed position of the clamps 90-92, wherein the distal end of the clamps 91-92 remains below the axis 18. In other words, in an embodiment of the invention, the movement of the clamps in the clamp mechanism is not symmetrical relative to the worm screw axis. In another embodiment of the invention, the movement of clamps in the clamp mechanism is symmetrical relative to the worm screw axis. In a particular embodiment, the pivot 98 of the clamp 90 (finger one or thumb) is in a position such that the movement of the clamp 90 is greater than the movement of the clamps 91-92.

In an embodiment of the invention, the separation distance, either the component in X and/or the component in Y, of the pivot 99 from the clamps corresponding to fingers two, three, four and/or five relative to the rotation axis 18 is greater than the separation distance of the pivot 98 from the clamp corresponding to finger one. In another embodiment of the invention, the separation distance, either the component in X and/or the component in Y, of the pivot 99 from the clamps corresponding to fingers two, three, four and/or five relative to rotation axis 18 is smaller than the separation distance of the pivot 98 from the clamp corresponding to finger one. In another embodiment of the invention, the separation distance, either the component in X and/or the component in Y, of the pivot 99 from the clamps corresponding to fingers two, three, four and/or five relative to the rotation axis 18 is equal to the separation distance of the pivot 98 from the clamp corresponding to finger one.

Therefore, when the movable displacement element 8 moves in one direction or in the opposite direction, the movable displacement element 8 urges the clamps into rotation relative to pivot 98 and 99, thus causing the clamps to open or close. Thus, the mechanical limit of said opening or closing occurs when the cylindrical protrusions 81 and 82 contact the corresponding ends of the slides 95 and 96 and/or when the clamps make physical contact with each other at the maximum closing points or at the maximum opening point. In a particular embodiment, electronic and/or electromechanical sensors stop the movement before such mechanical limits are reached. Therefore, the slides 95 and 96, in addition to providing one degree of freedom of displacement to the cylindrical protrusions 81 and 82, also allow the cylindrical protrusions 81 and 82 to rotate inside the slides 95 and 96, that is, the design of the slides 95 and 96 allows for the cylindrical protrusions 81 and 82 to have at least one degree of freedom of displacement and at least one degree of angular freedom relative to slides 95 and 96 respectively. In an embodiment of the invention, the slide 96 is an integral part of the clamp 91 and/or the clamp 92. In a particular embodiment, the clamp 91 is coupled to the clamp 92 at a height of the pivot 99 and the slide 96 is located at the proximal end of the clamp 92. In a particular embodiment, the coupling between the clamp 91 and the clamp 92 is a fixed coupling so that both clamps may move in the same way. In a particular embodiment, the coupling between the clamp 91 and the clamp 92 is a coupling at least partially allowing for rotation of one clamp relative to the other clamp, wherein a spring or elastic means is used to take both clamps 91 and 92 to a default position. One skilled in the art will appreciate that the location of the spring and/or the elastic return technique to provide for automatic return to a default position of the clamp 91 and/or the clamp 92 may vary without affecting the subject matter of the present invention.

Additionally, FIG. 10 shows the holes 17 defined by a plurality of holes along the fingers or clamps 90-92. The holes 17 allow other elements, such as cushions, gloves, tools, mechanical extensions, removable assemblies that provide a biological shape, etc., to be coupled to the clamps 90-92. One skilled in the art will appreciate that the number, configuration, dimensions, and location of such holes, either jointly or separately, may vary without affecting the subject matter of the present invention.

One skilled in the art will further appreciate that the separation distance of the pivot relative to the rotation axis may vary without affecting the subject matter of the present invention, wherein not only is the separation distance transverse to the rotation axis, but it is also a longitudinal separation distance to the rotation axis.

In an embodiment of the invention, the clamps 91 and 92 are fixed to each other. In an embodiment of the invention, the clamps 91 and 92 perform independent movements from each other. In a particular embodiment, the clamp 91 and/or the clamp 92 comprises an additional joint at the proximal end of the clamp 91 and/or the clamp 92, wherein said additional joint is free and restrained to allow for a predetermined angle of rotation of the corresponding clamp, and wherein said additional joint is coupled to at least one spring for returning thereof to its original position. In a particular embodiment, the restraint movement allows the clamp 91 and/or the clamp 92 to have an inclination of 0-25°. Additionally, in an embodiment of the invention, mechanical means are provided to adjust the tensile force of the clamp return spring. In a particular embodiment, the joint at the clamp proximal end comprises at least two degrees of freedom, wherein the degrees of freedom are angular.

In an embodiment of the invention the machining mark 62 formed on the plate 50 is located at and corresponds to the position of the pivot 99, and the machining mark 61 formed on the plate 50 is located at and corresponds to the position of the pivot 98, or vice versa.

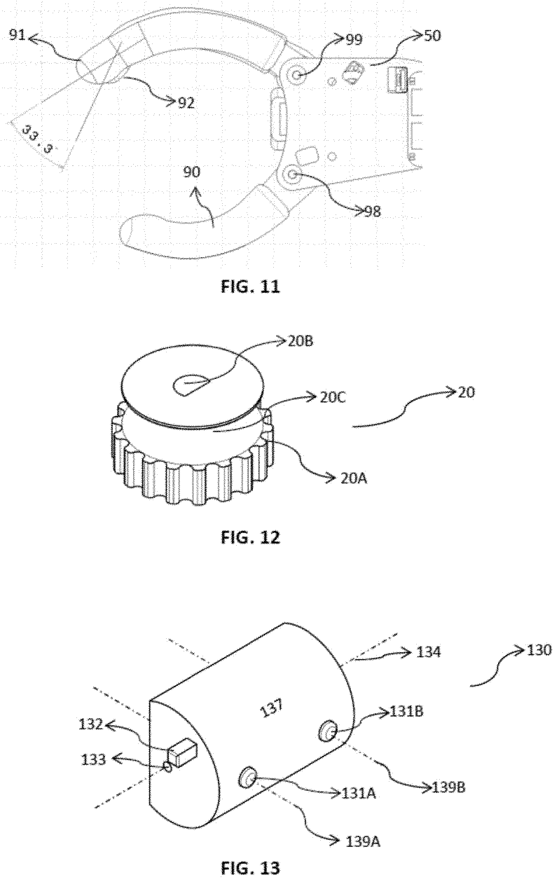

FIG. 11 shows a side view of an assembled prosthesis according to an embodiment of the present invention, wherein each of the fingers or clamps 90-92 are shown with a cover, wherein the cover can be a padded cover. Therefore, the configuration of the clamps 91 and 92 is shown, wherein the clamps 91 and 92 maintain a non-parallel default position. Thus, it can be observed how there is a difference or angle of separation of one distal end of the clamp 91 relative to the other distal end of the clamp 92 with an opening of about 33.3°±5. Thus, it can be observed how the concave or convex shape (depending on the point of reference) of the clamp 91 is at least partially different to that of the clamp 92. Additionally, it can be observed that clamps 91 and 92 have different lengths and/or reaches, with this being understood that, although the clamps 91-92 have the same length, the clamps 91-92 may have a different reach in view of their configuration, curvature, etc. Thus, in a particular embodiment of the invention, the clamps 91 has a greater reach than the clamp 92. In another embodiment, the clamp 91 has a smaller reach than the clamp 92. In another embodiment, each of the clamps 91-92 has the same reach.

FIG. 12 shows a drive gear 20 according to an embodiment of the invention. The drive gear 20 further comprises, in addition to the arrangement of teeth 20A and the coupling hole 20B, an adaptation 20 C allowing a belt to be coupled in order to transmit additional motion to other elements of the prosthesis. Therefore, one skilled in the art will appreciate that the belt adaptation 20C may vary in diameter, thickness, depth, profile, texture, etc. without affecting the subject matter of the present invention.

FIG. 13 shows an isometric view of a movable wrist element or link 130 defined by a link having at least one semicircular profile or convex face 137 including at least one hole through which a locking pin is passed (in this example, the locking pins 131A and 131B are shown), at least one tracking bar or activation button 132, and a clamping center 133 defining the rotation axis 134. In an embodiment of the invention, the movable wrist element 130 is used to provide a flexion/extension movement and/or an abduction/adduction movement to a hand prosthesis, regardless of the hand prosthesis type. One skilled in the art will appreciate that the number of locking pins, the location of the locking pins and the arrangement of the locking pins in the body of the movable wrist element 130 of one pin relative to another pin may vary without affecting the subject matter of the present invention. Additionally, the pin axis 139 (axis 139A and axis 139B for the pin 131A and the pin 131B, respectively) is shown, wherein each corresponding pin is linearly displaced. In an embodiment of the invention where at least two pins are provided, each pin 131 and each corresponding axis 139 are parallel to each other. In another embodiment of the invention, each pin 131 and each corresponding axis 139 are not parallel to each other, at least partially.

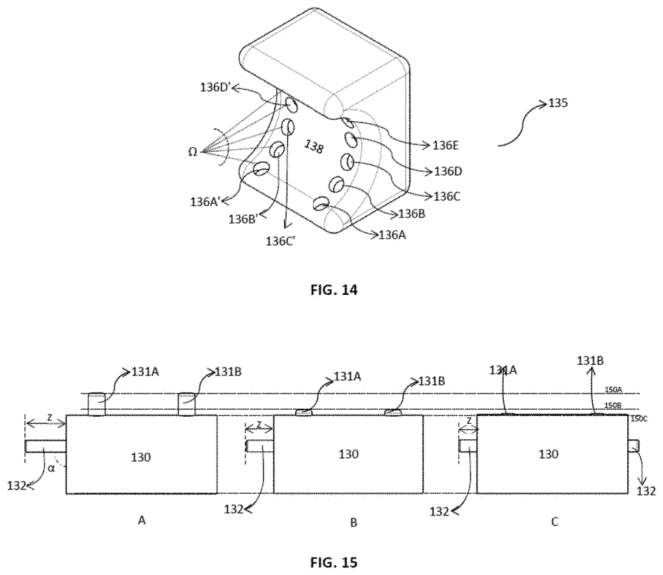

FIG. 14 shows an isometric view of a fixed wrist element or link 135 defined by a link having at least one semicircular profile or concave face 138 which is the counterpart of the movable wrist element 130 shown in FIG. 13, i.e. the convex shape of the movable wrist element 130 at least partially matches the concave shape of the fixed wrist element 135. The movable wrist element 130 is coupled to the fixed wrist element 135 by mechanical means known in the art (not shown in FIG. 14), wherein at least one degree of angular freedom is provided on the side of the movable wrist element 130 relative to the fixed wrist element 135, such as a hinge, bolt, pin, rivet, or any other mechanical means that provides an angular joint known in the art. In a particular embodiment, there is provided at least one degree of freedom corresponding to the transverse rotation or relative to the axis of said concave shape, that is, where one rotates relative to the other around the axis 134. Thus, the concave face of the fixed wrist element 135 has a plurality of holes 136 in its interior, wherein said holes are grouped together into groups of at least one hole defining a degree of inclination of the movable wrist element 130 relative to the axis 134. The fixed wrist element 135 of FIG. 14 shows the holes 136 grouped together into groups of two 136A and 136A′, 136B and 136B′, 136C and 136C′, 136D and 136D′, 136E and 136E′, and 136F and 136F′ (not shown in the isometric view). The holes 136 may vary in number and type of group without affecting the subject matter of the present invention. That is, this example shows six groups of holes 136, each group is arranged at an inclination or angle 4 different from the other, relative to the axis 134 and/or the axis of the concave shape of the fixed wrist element 135, and wherein the pin axes 139 and the corresponding pins 131 match one or more of the positions defined by Ω, such that when the pins 131 are moved to reach the interior of the corresponding holes 136, the degree of freedom of the movable wrist element 130 will be locked and prevented from rotating relative to the axis 134 since the pins 131 will be exposed to shear forces, which prevent angular movement, provided that the mechanical properties of the material exceed those of the shear forces involved between the pin 131, the hole of the movable wrist element 130 and the holes 136 of the fixed wrist element 135. The diameter, number and arrangement of the holes 136 per group may vary without affecting the subject matter of the present invention, provided that they are equivalent and/or match the diameter and arrangement of at least one pin 131. Additionally, the number of groups and their angle of separation between each other may vary without affecting the subject matter of the present invention, and the same angle of separation Ω between each adjacent group, or a different angle of separation Ω between each adjacent group of the holes 136 may be used.

One skilled in the art will appreciate that the shape and dimensions of the fixed wrist element 135 may vary without affecting the subject matter of the present invention, including the degree of curvature of the concave face of the fixed wrist element 135.

Thus, the movable wrist element 130 and the fixed wrist element 135 may have a locking relationship by means of at least one locking pin 131 and at least one locking hole 136. Therefore, the location, dimensions, number and arrangement of the locking holes 136 may vary depending on the arrangement, dimensions and number of locking pins 131, and the number of flexion/extension and/or abduction/adduction positions to be provided for the prosthesis. Additionally, a plurality of pairs of holes 136 are shown, which are arranged at different angles Ω. One skilled in the art will appreciate that the angle of separation Ω between each group of holes 136 may vary, and that the angle Ω may be the same or a different angle of separation relative to the adjacent, without affecting the subject matter of the present invention.

FIG. 15 shows a bottom view of the movable wrist element 130 in three different positions A, B and C which are activated by pressing the activation button 132 in a first direction, and wherein the activation button length is greater than at least one dimension of the movable wrist element 130, such that at least one of the activation button ends 132 protrudes from the body of the movable wrist element 130. In an embodiment of the invention, the portions A-C are deactivated by pressing the activation button 132 in a second direction, which is opposite to the first direction. In the position A, the movable wrist element 130 is in a position, with the locking pins 131A-131B being extended, i.e. the locking pins 131A-131B are projecting out of the body of the movable wrist element 130 and inserted into at least one hole 136 (not shown in FIG. 15). Thus, the movable wrist element 130 is locked, with the degree of inclination being defined by said at least one hole 136. In the position B the movable wrist element 130 is in the released position or free of rotation around the axis 134, with the locking pins being substantially inside the body of the movable wrist element 130 and outside the holes 136, and the locking pins are held in that position as long as the user keeps the activation button 132 pressed, so the movable wrist element 130 can be rotated relative to the axis 134 by the same user while pressing the activation button 132. Thus, when the activation button 132 is released, the pins in a projected or locked position automatically move to the locked position A to enter into the group of holes 136 corresponding to the inclination that has been assigned to the movable wrist element 130 when it was released. In the position C, the movable wrist element is in the maintained free position, with the pins being substantially inside the body of the movable wrist element 130, without being inserted in any hole 136, and wherein, even if the user releases the activation button 132, the pins are held in the maintained free position by mechanical fastening means known in the art, until said position is changed using the activation button 132. In an embodiment of the invention, the movable wrist element 130 is in a generally locked position, that is, the movable wrist element 130 default position is the position A.

Additionally, it can be observed that the positions A-C are controlled by the activation button 132 through mechanical means. Therefore, the activation button 132 generates from a horizontal movement a vertical movement in the corresponding pins. Additionally, the angle α defining the inclination relationship between the activation button 132 and the body of the movable wrist element 130 is also shown. Therefore, the design of the movable wrist element 130 may comprise an angle other than 90° so that the activation of the activation button 132 can be performed in a mostly anthropomorphic way. In a particular embodiment of the invention, the angle α may have a magnitude of 90°±20° (depending on whether it is a right hand or a left hand prosthesis). Additionally, one skilled in the art will appreciate that the inclination of the activation button 132 relative to the body of the movable wrist element 130 may be in any of the three planes.

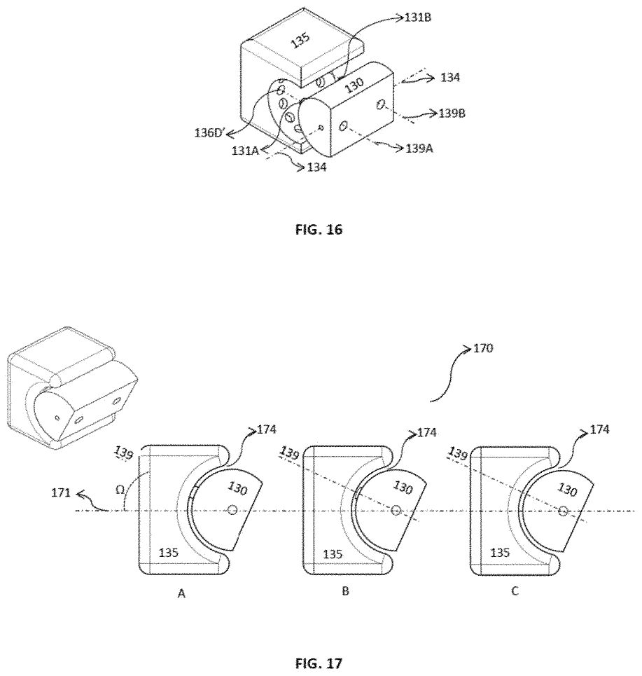

FIG. 16 shows an isometric view of the wrist elements 130 and 135 in a pre-assembled position, clearly illustrating the way of attachment thereof, wherein the shape and dimensions of the convex and concave portions of the wrist elements 130 and 135, respectively, allow for the wrist element 130 to be rotated relative to its axis 134 within the concave portion of the wrist element 135, and the arrangement of at least one pin 131 matches at least one of a plurality of holes 136 depending on the degree of inclination of the movable wrist element 130 and the number of wrist positions desired to be provided to the hand prosthesis, which may vary depending on the intended application.

In an embodiment of the invention, the rear face of the fixed wrist element 135 is coupled to the biological forearm or to a prosthesis of a patient, so that the concave portion of the fixed wrist element 135 is at the distal end of the arm. Thus, with the use of the mechanical means known in the art, such as one or a pair of parallel elongated fastening plates, the movable wrist element 130 is coupled to the fixed wrist element 135. Each of said plates (not shown in the Figs.) holds the fixed wrist element 135 at one of tis ends without providing any degree of freedom, but holds the movable wrist element 130 at its other end, thereby providing at least one degree of angular freedom or joint around the axis 134, such that the movable wrist element 130 is at the distal end of the patient or user's arm. One skilled in the art will appreciate that the way in which the rear face of the fixed wrist element is secured to the biological or prosthetic forearm of a patient or user may vary without affecting the subject matter of the present invention. Additionally, one skilled in the art will appreciate that the way in which the hand prosthesis is secured to the movable wrist element 130 may vary without affecting the subject matter of the present invention. In an embodiment of the invention, instead of one or a pair of elongated fastening plates between the wrist elements 130 and 135, the fixed wrist element 135 comprises side ends extending to hold the movable wrist element 130, providing one degree of angular freedom, which is parallel to the center of the concave shape or axis 134.

One skilled in the art will further appreciate that the number of pins 131 and holes 136, as well as its arrangement, dimensions and shape, may vary without affecting the subject matter of the present invention. Moreover, one skilled in the art will appreciate that the wrist arrangement disclosed herein is applicable for flexion/extension movements or for wrist abduction/adduction movements (radial and ulnar deviation). Furthermore, one skilled in the art will appreciate that a combination of two wrist arrangements disclosed herein allows for wrist flexion/extension and wrist abduction/adduction movements in a single apparatus.

One skilled in the art will further appreciate that the shapes and dimensions of the movable wrist element 130 and the fixed wrist element 135 may vary without affecting the subject matter of the present invention.

FIG. 17 shows an isometric view and three side views of an already assembled wrist arrangement 170 defined by a coupling of the movable wrist element 130 and the fixed wrist element 135 respectively, in three different positions A, B and C, and wherein the movable wrist element 130 has at least partially been rotated so that the pin axis 139 is different from a forearm axis 171 corresponding to the fixed position of the fixed wrist element 135 relative to the forearm of a user or patient. For illustrative purposes, the plate or connection element between the movable wrist element 130 and the fixed wrist element 135 is not shown. In the position A, the arrangement is in a locked position so that at least one of the pins 131 is within at least one of the corresponding set of holes 136, such that the joint around the axis 134 cannot be rotated unless one of the wrist elements is subjected to such an effort that the mechanical properties of the materials involved in this shear stress are overcome. Thus, this position in any of the angles chosen at the patient's discretion is useful when the patient is going to perform a specific operation such as holding a relatively light object, and wherein the inclination of the hand prosthesis is adjusted (i.e. the wrist is adjusted) to a desirable position. In the position B, the arrangement is in a released position, wherein the pins 131 are outside the holes 136, since the patient or user is pressing an activation button 132, and wherein the activation button is located in the movable portion, i.e. the activation button 132 is located in the movable wrist element 130, so it is easy for the user or patient to make the adjustment with a single biological hand so that, when the activation button is released, the assembled wrist 170 is automatically locked in the adjusted position. In the position C, the arrangement is in a maintained released position, such that the user stops pressing the activation button and the arrangement remains released so that the wrist can freely rotate until the position C is changed. In an embodiment of the invention, the shape of the fixed wrist element 135 defining the forearm axis 171 substantially corresponds to the longitudinal axis of the patient's biological or prosthetic forearm. In an embodiment of the invention, the forearm axis 171 maintains a default angle of inclination relative to the longitudinal axis of the patient's forearm.

Thus, in an embodiment of the invention, there is provided a prosthesis wrist mechanism, wherein said wrist mechanism provides at least one degree of angular freedom adjustable through mechanical means, wherein the adjustment mechanical means are located in the movable portion of the wrist mechanism. In a preferred embodiment, there is provided a wrist mechanism for the at least one degree of angular freedom of transverse rotation (flexion/extension) at different previously defined angles. The wrist mechanism comprising at least two operating positions: a locked position A and a released or free position B. In the locked position, the wrist mechanism is fixed at a certain angle relative to the axis. In the released position, the wrist is unlocked so it can freely rotate in any direction. In an embodiment of the invention, a torsion spring is coupled to the wrist angle of freedom, wherein the force exerted by the torsion spring is sufficient to position the wrist and the weightless hand prosthesis in a known and controlled position. That is, the force exerted by the torsion spring is opposite to the wrist movement so that, when the torsion spring is in the released position, the torsion spring remains in a controlled position, at least with a light weight. The released position is useful when a user or patient is moving and holding by gravity a heavy object, wherein the natural movement of the user causes the object to balance in a pendulum-like movement. In a locked position, said pendulum-like movement can break the elements that lock the position; therefore, to avoid this phenomenon, free rotation is at least partially allowed. In an embodiment of the invention, the free joint of the wrist comprises an elastic and/or damping means. In a particular embodiment, a torsion spring is coupled to said one degree of angular freedom (transverse rotation), wherein the torsion spring opposes to the movement. Additionally, in an embodiment of the invention, the activation button locking and/or unlocking the wrist mechanism is located at the distal end (relative to the biological portion of a user or patient) and/or the movable portion of the mechanism. In a particular embodiment, the activation button locking and/or unlocking the prosthesis is defined by an elongated part that can be pressed at any of its distal ends, and wherein its longitudinal axis is at least partially parallel to at least one rotation axis of said one degree of freedom of the wrist mechanism. In a particular embodiment, the longitudinal axis of the activation button 132 is substantially parallel to the rotation axis 134. The separation distance 174 between the movable wrist element 130 and the fixed wrist element 135 may vary without affecting the subject matter of the present invention, provided that the locking/unlocking or releasing relationship of the pins 131 with the holes 136 is allowed.

Thus, with the use of the wrist mechanism 170, a user is able to adjust the angle of inclination of the hand prosthesis using their biological hand (if any), wherein the release and locking for adjustment of the inclination is performed by a button located in the movable portion, that is, in the inclining portion.

Although the non-concave face of the movable wrist element is flat in shape, one skill in the art will appreciate that such shape may vary without affecting the subject matter of the present invention, and that said shape may include a design that facilitates its assembly with the corresponding hand prosthesis.

For purposes of illustration, some elements are not shown in some Figures, such as the activation button, the connection element between fixed wrist element 135 and the movable wrist 130, etc. without this affecting the scope of the present invention.

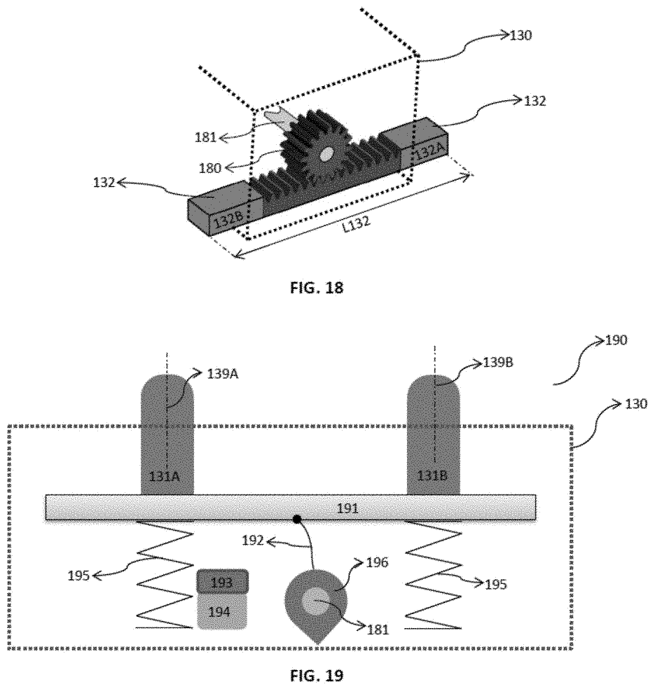

FIG. 18 shows an isometric view of the internal operation of the movable wrist element 130 where the activation button 132 is located according to an embodiment of the present invention. The activation button 132 is defined by an elongated bar having distal ends 132A and 132B that may or may not project out of the body of the movable wrist element 130, and at least one face located in its central portion, which includes a rack-like toothing. Additionally, there is shown a gear 180 having a shaft 181 in the center thereof, so that the gear teeth configuration and the gear rack configuration match each other. Thus, when the activation button 132 is pressed on one of its ends, the gear 180 and the shaft 181 are rotated in one direction or in an opposite direction depending on the end 132A or 132B that is pressed. The shaft 181 is coupled at each of its ends to the body of the movable wrist element 130 by mechanical means known in the art, such as a bearing, thus providing support to the movement and affecting friction to the minimum. One skilled in the art will appreciate that the length L132 of the activation button 132 may vary without affecting the subject matter of the present invention, such that, depending on the use or preference of the user, the activation button 132 may have in its position A, B or C a first distal end 132A, a second distal end 132B and/or both activation button ends 132 at sight, i.e. said first distal end 132 and said second distal end 132B projecting out of the body of the movable wrist element 130 in any of the positions A, B and/or C.

FIG. 19 shows a pin deployment mechanism 190 located inside the body of the movable wrist element 130. The pin deployment mechanism 190 allows for deployment and contraction of the pins 131 according to an embodiment of the present invention. Thus, the at least one pin 131 (in this case the pins 131A and 131B), a metal plate 191 holding the pins 131, at least one spring 195 for each pin 131 and/or for each plate 191, at least a magnetic means 193, such as a magnet, a base or supporting means 194 for the magnetic means 193, a cord 192, and a cam 196 coupled to the shaft 181 so that said cam 196 is rotated by the shaft 181, are shown. Therefore, when the shaft 181 is rotated by pressing the activation button 132, the cord 192 is wound or unwound (depending on the direction of the rotation) around the shaft 181 so when the cord 192 is wound, the cord 192 pulls down the metal plate 191 to an unlocked position of the pins 131 (position B), with the pins 131 being inserted into the body of the movable wrist element 130 through its holes (not shown in FIG. 19), such that if the button is no longer pressed, the springs 195 will push up the metal plate 191 together with the pins 131 thus unwinding the cord 192 so that pins 131 will return to a locked position (position A). By pressing the activation button 132, the shaft 181 is rotated, thus winding the cord 192 and pulling down the metal plate 191 and the pins 131, such that if the activation button 132 is pressed for a sufficient time, the metal plate 191 will contact the magnetic means 193 to hold the metal plate 191 and the pins 131 by magnetic force, so that when the activation button 132 is no longer pressed, the unlocked or released position (position C) will remain fixed until the metal plate 191 stops contacting the magnetic means 193. The contraction depth of the pins 131 may be adjusted by the height at which the base 194 holding the magnetic means 193 is placed or through another technique in the art. One skilled in the art will appreciate that the mechanical properties of the springs 195, such as the Young's modulus, material, etc., may vary without affecting the subject matter of the present invention, and that the force exerted on the plate by the springs 195 must be less than the magnetic force with which the magnet holds the metal plate 191. Thus, by pressing the activation button 132 in a reverse direction, the shaft 181 rotates together with the cam 196 mechanically pushing the metal plate 192 to separate it from the magnet 193 and then the springs will automatically push the metal plate 191 together with the cord, and the cord will be unwound while the metal plate 191 rises to a mechanical limit in the locking position of the pins 131.

In an embodiment of the invention, an operating pattern of the wrist mechanism is provided:

| Initial | Final | |
|---|---|---|
| position | position | Operation in the activation button |
| A | B | Press end 1 until it is unlocked and keep it pressed |
| B | C | Press end 1 to the limit and then release it |
| A | C | Press end 1 in a single movement to the limit |
| B | A | Release |
| C | B | Press end 2 of the activation button until it is |
| | | unlocked while slightly pressing end 1 |
| C | A | Press end 2 of the activation button until it is |
| | | unlocked and then release it |

*Note:
In any position B or C, the user can adjust the wrist inclination.

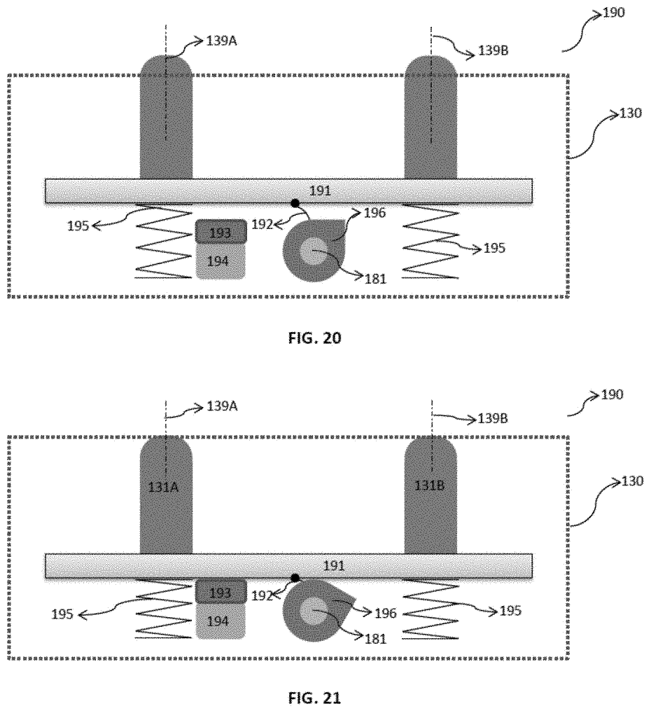

FIG. 20 shows the mechanism 190 in an unlocked position of the pins 131 (position B), as the user presses and keeps the button 132 pressed, causing the cord 192 to wind when the shaft 181 is rotated, wherein the magnetic plate is not in contact with the magnetic means 193. Thus, when the user releases the activation button 132, the springs 195 push the pins outwardly to a locked position (position A). In an embodiment of the invention, the shaft 181 has at least two different diameters in its length, with at least one of those diameters defining a perimeter so that the cord 181 is wound either faster (larger diameter) or slower (smaller diameter). In an embodiment of the invention, the diameter of the shaft 181 is such that its perimeter is proportional to at least one of the list of: a toothed relationship of gear 180 with the rack of the activation button 132, the distance between the metal plate 191 and the magnetic means 193, the activation distance Z of the activation button 132 and/or combinations thereof.

FIG. 21 shows the mechanism 190 in a maintained unlocked or fixed position of the pins (position C), wherein the magnetic plate 191 is in contact with the magnetic means 193. Thus, when the activation button 132 is pressed so that the cam 196 comes into contact with the plate 191, the plate 191 ceases to contact the magnetic means 193.

One skilled in the art will appreciate that the technique to generate magnetic force through the magnetic means 193 may vary without affecting the subject matter of the present invention, and that a ferrous magnet, a neodymium magnet, an electromagnet, etc. may also be used. One skilled in the art will further appreciate that the dimensions and/or configuration and thus the magnetic attraction of said magnetic means may vary without affecting the subject matter of the present invention. In an embodiment of the invention, the metal plate 191 is held in the position C by mechanical means, such as a spring-loaded hook mechanism or any other mechanism known in the art.

In a particular embodiment, an engagement by a magnetic means is used to magnetically fix the parts of the prosthesis in a desired position. One skilled in the art will appreciate that the shape, dimensions, magnetic attraction and location of the magnetic means may vary without affecting the subject matter of the present invention, and that ferrous magnets, neodymium magnets, electromagnets and/or combinations thereof may also be used, and that different materials with a magnetic capacity are already known in the art are used.

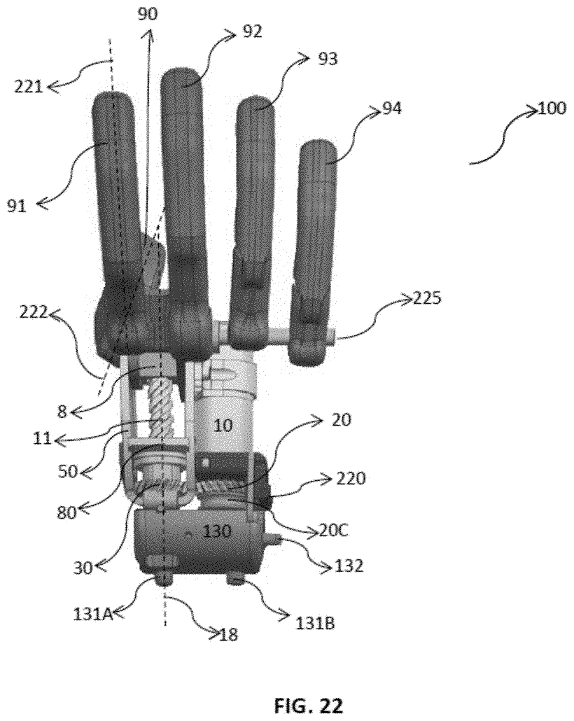

FIG. 22 shows a top view of a right-hand prosthesis assembly with a movable wrist element 130 according to an embodiment of the invention, which comprises: a movable wrist element 130, at least five clamps or fingers 90-94; at least one actuator 10 coupled by a gear 20 and 30 to a worm screw mechanism 11 located inside the plate 50, and the movable wrist element 130 is coupled by fastening means between the plate 50 and the motor 10, wherein the pins 131A and 131B, etc. are also shown. Thus, it can be observed how fingers one 90, two 91, and three 92 are directly driven by a movable displacement element 8 having ends located within the longitudinal flutes 52A and 52A′ (not shown in FIG. 22) of the plate 50, wherein the mobile displacement element 8 is in turn driven by the worm screw 11 coupled to an actuator 10 by a gear 20 and 30. Therefore, in an embodiment of the invention, the finger four 93 and finger five 94 are provided, which are articulately coupled by the joint 225 to the fingers 91 and 92. In a particular embodiment, finger four 93 and finger five 94 are flexed by a mechanism having at least one band (not shown in the Figs.) and/or flexible rubber band coupled at the adaptation 20C of gear 20, passing through roller 220, to at least one of the fingers 93 and/or 94, such that in a first direction of rotation of the gear 20, the fingers 93 and 94 are flexed. In an embodiment of the invention, the fingers 93 and 94 are extended when the gear 20 is rotated in a second direction. In an embodiment of the invention, the fingers 93 and 94 are extended by a spring mechanism coupled to the base of the fingers 93 and 94. In a particular embodiment, the spring mechanism of the fingers 93 and 94 is defined by at least one torsion spring coupled at one of its end to at least one of fingers 93 and/or 94 and at the other end to the prosthesis body and/or to the joint 225. Additionally, the plane 221 on which the finger two 91 moves along its flexion and/or extension path is shown. Moreover, the plane 222 on which the finger one 90 is arranged is also shown. Thus, it can be observed how the plane 222 has a certain degree of inclination relative to the plane 221 in order to provide an anthropomorphic image. In a particular embodiment, the plane 222 is substantially parallel to the plane 221. In an embodiment of the invention, the body of the finger one 90 includes an inclination, although its flexion and/or extension movement is substantially parallel to the plane 221.

Additionally, the operating axis 18, the axis 221 of the finger 91, which is substantially parallel to the axis of the fingers 92, 93 and 94, and the axis 220 having an angle of inclination relative to the axis 221 are shown. In an embodiment of the invention, the separation angle between the axes 221 and 222 is 25°±5°. In an embodiment of the invention, the clamps 90 and 91 are aligned with each other, that is, the movement of clamps 90 and 91 is performed on the same plane. In a particular embodiment, the clamps 90 and 91 are moved on different but at least partially converging planes in the closed position.

In an embodiment of the invention, the retainer 80 is defined by a PCB electronic board. In a particular embodiment, the PCB electronic board is an electronic board with a thickness of at least 3.175 mm.

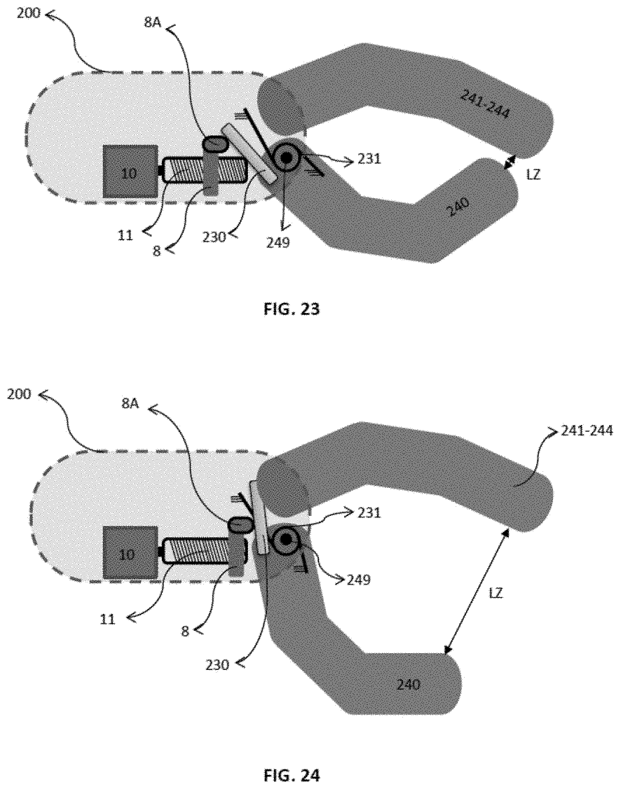

FIG. 23 shows a side view of a hand prosthesis in a closed finger position according to another embodiment of the invention. Thus, a clamp or upper finger corresponding to fingers two 241, three 242, four 243 and five 244 is illustrated (only the finger two 241 in the side view is shown); an articulated portion defined by at least one finger one 240 articulated by a joint providing at least one degree of angular freedom 249; an actuator 10, a worm screw 11 coupled to the actuator 10; a prosthesis body 200 accommodating several prosthesis elements; a movable displacement element 8 coupled to the worm screw 11 and the prosthesis body 200 to restrain its rotation relative to the worm screw 11, wherein the movable displacement element 8 comprises a follower 8A, at least one tracking bar 230, and at least one automatic return mechanical means 231. In an embodiment of the invention, the automatic return mechanical means 231 is a spring coupled to the joint 249, between the articulated portion 240 and the prosthesis body 200, such that the spring is held, at one end, by the prosthesis body 200 and, at the other end, by the articulated finger 240. In a particular embodiment, the spring is coupled when the articulated finger is in a flexed position. In an alternative embodiment, the spring is coupled when the articulated finger is in an extended position. In an embodiment of the invention, the fingers 241-244 are fixed, that is, the fingers do not comprise phalanges or joints. Thus, when the actuator 10 is activated, the actuator causes the worm screw 11 to be rotated, which in turn causes the movable displacement element 8 to be moved with a force greater than the force of the automatic return mechanical means 231, such that when the follower 8A comes into contact with the tracking bar 230, the follower 8A moves together with the movable displacement element 8 causing the articulated portion 240 to rotate relative to the joint 249.

The prosthesis body 200 may include a design and/or shape similar to a biological hand palm, wherein the adjustment of the scale of the prosthesis body 200 and the fingers 241-244 makes it possible for the prosthesis to be used in children of different ages or growth stages.

FIG. 24 shows the prosthesis of FIG. 23 in an open finger position, wherein the actuator has caused the movable displacement element 8 to be moved together with the tracking bar 230, such that the articulated portion 240 is moved to an open finger position when the articulated portion 240 is pivoted around the axis 249. The opening capacity LZ of said finger relative to the fingers 241-244 may vary depending on the displacement of the movable displacement element 8, the arrangement of the tracking bar 230 relative to the body of the finger 240, the location of the joint 249, the dimensions of the follower 8A and/or combinations thereof. Thus, when the actuator 10 rotates in a reverse direction, the movable displacement element 8 is also moved in the reverse direction without the movable displacement element 8 pulling the tracking bar 230, i.e. a force is exerted by the automatic return mechanical means 231 that causes the finger 240 to return to a closed or flexed position without the actuator 10 exerting a force on said flexion. In an embodiment of the invention, the return speed of the movable displacement element 8 may be adjusted to regulate the speed at which the hand prosthesis flexes.

Thus, a hand prosthesis is provided which comprises an actuator providing the extension movement and an elastic mechanism providing the flexion movement, wherein the speed at which the flexion and/or extension is performed is regulated by the rotation speed of the actuator. In an alternative embodiment, the actuator provides the flexion movement and the elastic mechanism provides the extension movement, wherein the speed at which the flexion and/or extension is performed is regulated by the rotation speed of the actuator. One skilled in the art will appreciate that the actuator 10 can generate a flexion and/or extension movement, either by pushing and/or pulling the tracking bar 230 without affecting the subject matter of the present invention.

In an embodiment of the invention, the follower 8A comprises a bearing and/or an electronic push button to perform an operation. One skilled in the art will further appreciate that the shape of the follower 8A may vary without affecting the subject matter of the present invention.

In an embodiment of the invention, the hole 60 is drilled or perforated before the plate 50 has been folded. In an embodiment of the invention, the hole 60 is drilled after the plate 50 has been folded.

In an embodiment of the invention, the axis of the worm screw 11 and the shaft of the motor 10 have a separation angle. In a particular embodiment, a transmission system between non-parallel axes is used for the transmission of motion between the non-parallel worm screw 11 and the non-parallel shaft of the motor 10.

One skilled in the art will appreciate that the term “longitudinal” refers to the axis of a body in a direction parallel to the side or larger dimension of said body.

One skilled in the art will further appreciate that the type of motor and/or actuator used in the embodiments of the invention disclosed herein may vary without affecting the subject matter of the present invention, and that one of a stepper motor, a direct current motor, an alternate current motor, a servomotor, brush or brushless motors, geared motor, etc. may be selected.

Moreover, one skilled in the art will appreciate that the number and configuration of the strings of the worm screw 11, including the thread of the movable displacement element 8 may vary without affecting the subject matter of the present invention.

Therefore, in an embodiment of the invention a hand prostheses assembly is claimed. The hand prosthesis assembly comprising an elongated plate having a plurality of machining marks, wherein the plate has been folded into a “U” shape having a substantially circular hole in the central portion of the curvature of the already folded plate, the plate comprising at least one longitudinal flute for each inner face of the U-shaped plate, wherein said at least one flute is parallel to a rotation axis defined by a line transversally passing through the center of said substantially circular hole; an electric motor coupled adjacent to an outer face of the U-shaped plate, such that the motor shaft is parallel and not concentric to the rotation axis, and wherein said shaft is pointing to the proximal end of the prosthesis assembly; a worm screw coupled to the plate, such that the axis of the worm screw is parallel and concentric to the rotation axis, and wherein the tip of the worm screw is pointing to the distal end of the prosthesis assembly; at least one mechanical transmission transmitting the motion from the motor shaft to the worm screw; a stiff movable element having a hole with a matching thread and coupled to the worm screw, wherein each side of said stiff movable element comprises a cylindrical protrusion with a projection, and wherein each protrusion is at least partially inserted into each corresponding flute of the U-shaped plate; at least a first elongated clamp comprising at its proximal end a groove that is coupled to one of the cylindrical protrusions of the stiff movable element, the middle portion of said stiff movable element comprising a first hole that is coupled to a first corner of the distal end of the plate; and at least two second substantially parallel elongated clamps joined together, wherein at least one clamp comprises at its proximal end a groove that is coupled to another of the cylindrical protrusions of the stiff movable element, and the middle portion of said stiff movable element comprising a second hole that is coupled to a second corner of the distal end of the plate, and wherein the relationship of the first clamp with the at least two second clamps is a concave-convex clamp relationship.

Additionally, at least one method of manufacturing a frame and/or partial support of a left or right hand prosthesis with the use of an elongated plate having a front and a rear face is claimed. The method comprising the steps of forming a first plurality of machining marks on a first side of the plate relative to the traverse axis of symmetry, the first plurality of machining marks comprising at least one longitudinal flute and at least one transverse flute; forming a second plurality of machining marks on a second side of the plate relative to the transverse axis of symmetry, the second plurality of machining marks comprising at least one longitudinal flute and at least one transverse flute, wherein the second plurality of machining marks has a mirror relationship with the first plurality of machining marks; making at least one fold in the elongated plate to provide a “U” shape, thus defining lateral walls and a central portion thereof; and making a hole in the central portion of the “U” shape, wherein the longitudinal flute of the first plurality of machining marks and the longitudinal flute of the second plurality of machining marks are parallel to an axis transversely passing through the center of the hole.

Moreover, a robotic joint is claimed. Said robotic joint comprising at least one application as wrist prosthesis for gripping an object by gravity with at least one adjustable degree of angular freedom comprised by a fixed link comprising a plurality of groups of holes, wherein the group of holes defines a degree of inclination of the prosthesis; a movable link comprising at least one extensible pin which when inserted in at least one of the plurality of holes, causes the prosthesis to be locked in the position defined by the location of said at least one hole, and wherein the movable link rotates around an rotation axis relative to the fixed link; a mechanical button coupled to the movable link, said mechanical button having at least two positions which, when pressed and remained in a first position, causes the pin to be contracted by a first distance, and wherein when the button is pressed and remained in a second position, causes the pin to be contracted by a second distance, with the second distance being greater than the first distance; and at least one compression spring coupled to the base of at least one pin, such that a constant force is exerted by the spring, thus projecting out said at least one pin; wherein, when the second distance is contracted, the pin is fastened by mechanical means coupled to the body of the movable link, wherein the force exerted by said mechanical means is greater than the force exerted by at least one compression spring.

Furthermore, in an embodiment of the invention a hand prosthesis is claimed. Said hand prosthesis comprising a prosthesis body; at least a first group of prosthesis fingers fixedly coupled to the prosthesis body; at least one articulated finger coupled to the prosthetic body by a joint, wherein said joint provides at least one degree of angular freedom; at least one actuator comprising a mechanism having a displacement movable element, such that when the actuator is activated, the actuator causes the movable displacement element to linearly move; a tracking bar coupled to the articulated finger in such a position that, when the tracking bar is pushed, the articulated finger becomes articulated; a spring rotating around its axis coupled to the joint of the articulated finger, such that the spring is held at one end by the prosthesis body and at the other end by the articulated finger; wherein the displacement movable element, when moved in a first direction, causes the tracking bar to be pushed and when moved in a second direction prevents the tracking bar from being pushed.

The foregoing description of the various embodiments has been presented only for purposes of illustration and description. It is not intended to be exhaustive or to limit the invention to the precise forms disclosed herein. Therefore, many modifications and variations will become apparent to those skilled in the art. Moreover, the foregoing disclosure is not intended to limit the present invention.

1. A hand prosthesis assembly comprising:
an elongated plate having a plurality of machining marks, wherein the elongated plate has been folded into a “U” shape having a substantially circular hole in the central portion of the curvature, said elongated plate comprising at least one longitudinal flute for each inner face of the “U” shape, wherein said at least one flute is parallel to a rotation axis defined by a line transversely passing through the center of said substantially circular hole;
an electric motor coupled adjacent to an outer face of the U-shaped plate, such that the motor shaft is parallel to and not co-linear with the rotation axis, and wherein the shaft is pointing to the proximal end of the prosthesis assembly;
a worm screw coupled to the plate, such that the axis of the worm screw is parallel to and co-linear with the rotation axis, and wherein the tip of the worm screw is pointing to the distal end of the prosthesis assembly;
a mechanical transmission transmitting the motion from the motor shaft to the worm screw;
a stiff movable element having a hole with a matching thread and coupled to the worm screw, wherein said stiff movable element comprises a cylindrical protrusion with a projection on each side thereof, and wherein each protrusion is at least partially inserted into each corresponding flute of the U-shaped plate;
at least one first elongated clamp comprising at its proximal end a groove that is coupled to one of the cylindrical protrusions of the movable element, and comprising in its middle portion a first hole that is coupled to a first corner of the distal end of the plate; and
at least two second substantially parallel elongated clamps joined together, at least one of said at least two second substantially parallel elongated clamps joined together comprising at its proximal end a groove that is coupled to another of the cylindrical protrusions of the movable element, and comprising in its middle portion a second hole that is coupled to a second corner of the distal end of the plate, and wherein the relationship of the first clamp with the second clamps is a concave-concave clamp relationship relative to the rotation axis.

2. The assembly according to claim 1, wherein the mechanical motion transmission is defined by a set of gears, said set of gears comprising a drive gear and a driven gear.

3. The assembly according to claim 2, wherein the set of gears is defined by spur gears with helical teeth.

4. The assembly according to claim 2, wherein the driven gear is coupled between the base of the worm screw and the substantially circular hole.

5. The assembly according to claim 4, wherein the driven gear is coupled to the substantially circular hole by means of a radial or combined bearing.

6. The assembly according to claim 2, wherein the gears of the set of gears have the same diameter.

7. The assembly according to claim 1, wherein coupling of the proximal end either of the first clamp or the second clamps provides at least one degree of angular freedom and one degree of freedom of displacement.

8. The assembly according to claim 1, wherein the plurality of machining marks is formed on a first side and on a second side of the plate relative to the transverse axis of symmetry, in a mirror configuration.

9. The assembly according to claim 1, wherein the plurality of machining marks further comprises at least one machining mark selected from the list of: drilling, slotting, hollowing and/or combinations thereof.

10. The assembly according to claim 1, wherein coupling of the middle portion either of the first clamp or the second clamps provides at least one degree of angular freedom.

11. The assembly according to claim 1, wherein the machining marks formed on a first side of the elongated plate are the same as the machining marks formed on a second side of the elongated plate, in a mirror relationship.

12. The assembly according to claim 1, wherein the separation distance between the first hole and the rotation axis is greater than the separation distance between the second hole and the rotation axis.

13. The assembly according to claim 1, wherein the separation distance between the first hole and the rotation axis is smaller than the separation distance between the second hole and the rotation axis.

14. The assembly according to claim 1, wherein the separation distance between the first hole and the rotation axis is the same as the separation distance between the second hole and the rotation axis.

15. The assembly according to claim 1, wherein the angle of separation between the longitudinal axis of the distal end of a second clamp and the longitudinal axis of the distal end of another second clamp is 33.3°±5.

16. The assembly according to claim 2, wherein a retainer is coupled to the U-shaped plate by at least a transverse flute located on the plate, and wherein the retainer comprises a hole through which the worm screw is passed.

17. The assembly according to claim 16, wherein a bearing is coupled between the distal end of the driven gear and the retainer.

18. The assembly according to claim 16 wherein the retainer is defined by a PCB card comprising a part of the electronics of the prosthesis assembly.

19. The assembly according to claim 1, wherein the assembly further comprises at its proximal end a wrist assembly defined by:
a movable link coupled to the proximal end of the hand assembly, wherein the movable link comprises at least one extensible pin partially extended out of the body of the movable link by elastic means, and contracted inside the body of the movable link;
a fixed link coupled on one side to a user's forearm and coupled on the other side to the movable link, providing the movable link with at least one degree of angular freedom, wherein the fixed link comprises a plurality of holes located at different angles relative to the longitudinal axis of the forearm, wherein said at least one pin is at least partially inserted to block said at least one degree of angular freedom;
an activation mechanism coupled to the movable link, said activation mechanism comprising an elongated button within the movable link, wherein said elongated button length is greater than the body of the movable link, such that at least one of the ends of the elongated button extends out of the body of the movable link, wherein said elongated button, when partially pressed on a first button end protruding from the body of the movable link, causes the at least one pin to be contracted, thereby unlocking the at least one degree of angular freedom, and wherein the elongated button, when completely pressed on the same first button end, causes the at least one pin to be contracted, thereby unlocking the at least one degree of angular freedom and said at least one pin is held in said contracted position by a mechanical means, thus defining a held pin position.

20. The assembly according to claim 19, wherein the mechanical means holding the at least one pin is a magnetic means defined by at least one magnet.

21. The assembly according to claim 19, wherein the button, when at least partially pressed on a first button end, causes the second button end to protrude from the body of the movable link.

22. The assembly according to claim 21, wherein the second button end, when pressed, causes the at least one pin to be released from its held position.

23. A method of manufacturing a partial support of left or right hand prosthesis with the use of an elongated plate having front and rear faces, said method comprises the steps of:
forming a first plurality of machining marks on a first side of the plate relative to the transverse axis of symmetry, wherein the first plurality of machining marks comprises at least one longitudinal flute and at least one transverse flute;
forming a second plurality of machining marks on a second side of the plate relative to the transverse axis of symmetry, wherein the second plurality of machining marks comprises at least one longitudinal flute and at least one transverse flute;
wherein the second plurality of machining marks has a mirror relationship with the first plurality of machining marks;
making at least one fold in the elongated plate to provide a “U” shape, thus defining side walls and a central portion thereof; and
making a hole in the central portion of the “U” shape;
wherein the longitudinal flute of the first plurality of machining marks and the longitudinal flute of the second plurality of machining marks are parallel to an axis transversally passing through the center of the hole.

24. The method according to claim 23, wherein the method further comprises the step of
forming at least one machining mark selected from the list of: drilling, slotting, hollowing and/or combinations thereof.

25. The method according to claim 23, wherein the step of making a hole is carried out before the step of making at least one fold.

26. The method according to claim 23, wherein the first plurality of machining marks and the second plurality of machining marks are formed on the front face and/or the rear face of the elongated plate.

27. The method according to claim 23, wherein the elongated plate is made of an aluminum alloy.
